(19) United States
(12) Patent Application Publication
(10) Pub. No.: US 2010/0035951 A1
(43) Pub. Date: Feb. 11, 2010

(54) 2-AMINO BENZIMIDAZOLE DERIVATIVES AND THEIR USE AS MODULATORS OF SMALL-CONDUCTANCE CALCIUM-ACTIVATED POTASSIUM CHANNELS

(75) Inventors: Ulrik Svane Sørensen, Soborg (DK); Birgitte L. Eriksen, Farum (DK); Lene Teuber, Vaerlose (DK); Dan Peters, Malmo (SE); Dorte Strøbaek, Farum (DK); Tina Holm Johansen, Smorum (DK); Palle Christophersen, Ballerup (DK)

Correspondence Address: BIRCH STEWART KOLASCH & BIRCH, PO BOX 747, FALLS CHURCH, VA, 22040-0747, US

(73) Assignee: NEUROSEARCH A/S, Ballerup (DK)

(21) Appl. No.: 12/307,083

(22) PCT Filed: Jul. 5, 2007
(86) PCT No.: PCT/EP2007/056826

§ 371 Date: Dec. 30, 2008

Related U.S. Application Data
(60) Provisional application No. 60/818,841, filed on Jul. 7, 2006.

(30) Foreign Application Priority Data
Jul. 7, 2006 (DK) PA 2006 00944

(51) Int. Cl. A61K 31/4184; C07D 235/00

(52) U.S. Cl. 514/395; 548/307.4

(57) ABSTRACT

This invention relates to 2-amino benzimidazole derivatives of Formula 1a or 1b:

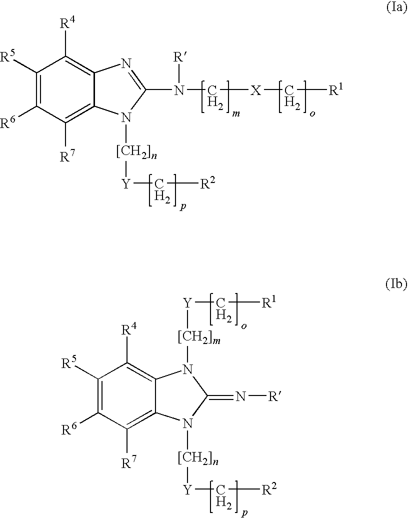

which are useful as modulators of small-conductance calcium-activated potassium channels (SK channels). In other aspects the invention relates to the use of these compounds in a method for therapy and to pharmaceutical compositions comprising the compounds of the invention.

### TECHNICAL FIELD

This invention relates to novel 2-amino benzimidazole derivatives useful as modulators of small-conductance calcium-activated potassium channels (SK channels). In other aspects the invention relates to the use of these compounds in a method for therapy and to pharmaceutical compositions comprising the compounds of the invention.

### BACKGROUND ART

Three subtypes of small-conductance calcium-activated potassium channels (SK channels) have been cloned: SK1, SK2 and SK3 (corresponding to KCNN1-3 using the genomic nomenclature). The activity of these channels is determined by the concentration of free intracellular calcium ([Ca²⁺]ᵢ) via calmodulin that is constitutively bound to the channels. SK channels are tightly regulated by [Ca²⁺]ᵢ in the physiological range being closed at [Ca²⁺]ᵢ up to around 0.1 μM but fully activated at a [Ca²⁺]ᵢ of 1 μM. Being selective for potassium, open or active SK channels have a hyperpolarizing influence on the membrane potential of the cell. SK channels are widely expressed in the central nervous system. The distribution of SK1 and SK2 show a high degree of overlap and display the highest levels of expression in neocortical, limbic and hippocampal areas in the mouse brain. In contrast, the SK3 channels show high levels of expression in the basal ganglia, thalamus and the brain stem monoaminergic neurons e.g. dorsal raphe, locus coeruleus and the ventral tegmental area (Sailer et al.: Comparative immunohistochemical distribution of three small-conductance Ca²⁺-activated potassium channel subunits, SK1, SK2, and SK3 in mouse brain; Mol. Cell. Neurosci. 2004 26 458-469). The SK channels are also present in several peripheral cells including skeletal muscle, gland cells, liver cells and T-lymphocytes.

The hyperpolarizing action of active SK channels plays an important role in the control of firing pattern and excitability of excitable cells. SK channel inhibitors such as apamin and bicuculline-methobromide have been demonstrated to increase excitability whereas the opener 1-EBIO is able to reduce electrical activity. In non-excitable cells where the amount of Ca²⁺ influx via voltage-independent pathways is highly sensitive to the membrane potential an activation of SK channels will increase the driving force whereas a blocker of SK channels will have a depolarising effect and thus diminish the driving force for calcium.

Based on the important role of SK channels in linking [Ca²⁺]ᵢ and membrane potential, SK channels are an interesting target for developing novel therapeutic agents.

WO 03/094861 describes bis-benzimidazoles and related compounds as potassium channel modulators.

A review of SK channels and SK channel modulators may be found in Liegeois J-F et al.: Modulation of small conductance calcium-activated potassium (SK) channels: a new challenge in medicinal chemistry; Current Medicinal Chemistry 2003 10 625-647.

Known modulators of SK channels suffer from being large molecules or peptides (apamin, scyllatoxin, tubocurarine, dequalinium chloride, UCL1684) or having low potency (1-EBIO, riluzole). Thus, there is a continued need for compounds with an optimized pharmacological profile. In particular, there is a great need for selective ligands, such as SK3 channel modulators.

WO 00/01676 describes novel potassium channel blocking agents.

U.S. Pat. No. 3,981,886, U.S. Pat. No. 4,004,016, Yale H L & Bristol J A; Journal of Heterocyclic Chemistry 1978 15 (3) 505-7, and Settimo et al; Farmaco 1994 49 (12) 829-34 describe amino-benzimidazole derivatives useful as anti-inflammatory agents.

### SUMMARY OF THE INVENTION

In its first aspect, the invention provides a 2-amino benzimidazole derivative of Formula Ia or Ib:

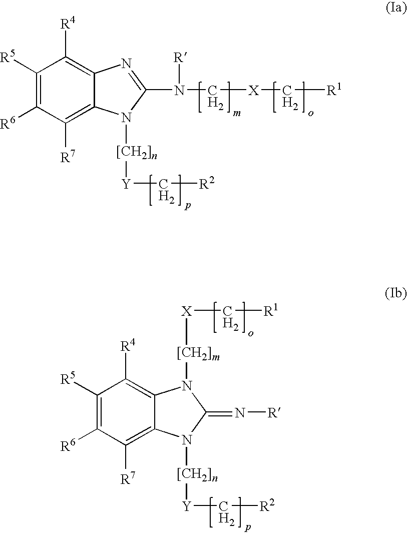

or any of its isomers or any mixture of its isomers, or a pharmaceutically acceptable salt thereof, wherein m, n, o, p, X, Y, R′, R¹, R², R⁴, R⁵, R⁶ and R⁷ are as defined below.

In its second aspect, the invention provides a pharmaceutical composition, comprising a therapeutically effective amount of a compound of the invention, or any of its isomers or any mixture of its isomers, or a pharmaceutically acceptable salt thereof, together with at least one pharmaceutically acceptable carrier, excipient or diluent.

In a further aspect, the invention provides the use of a compound of the invention, or any of its isomers or any mixture of its isomers, or a pharmaceutically acceptable salt thereof, for the manufacture of a pharmaceutical composition for the treatment, prevention or alleviation of a disease or a disorder or a condition of a mammal, including a human, which disease, disorder or condition is responsive to modulation of SK channels.

In a still further aspect, the invention relates to a method for treatment, prevention or alleviation of a disease or a disorder or a condition of a living animal body, including a human, which disorder, disease or condition is responsive to modulation of SK channels, which method comprises the step of administering to such a living animal body in need thereof a therapeutically effective amount of a compound of the invention, or any of its isomers or any mixture of its isomers, or a pharmaceutically acceptable salt thereof.

Other objects of the invention will be apparent to the person skilled in the art from the following detailed description and examples.

### DETAILED DISCLOSURE OF THE INVENTION

### 2-amino benzimidazole derivatives

In its first aspect the present invention provides 2-amino benzimidazole derivatives of formula Ia or Ib:

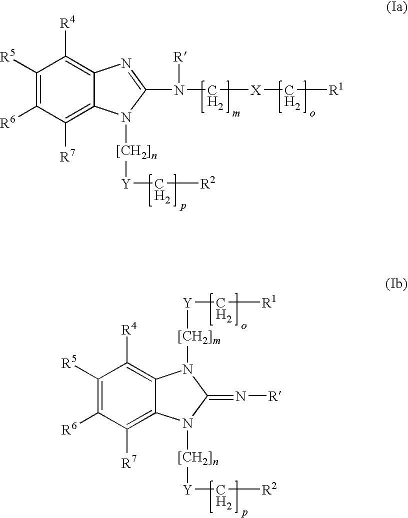

or any of its isomers or any mixture of its isomers, or a pharmaceutically acceptable salt thereof, wherein

m is 0, 1 or 2;

n is 0, 1 or 2;

o is 0, 1 or 2;

p is 0, 1 or 2;

X and Y, independently of each other, represent CH₂, S, O or NR″; wherein R″ represents hydrogen or alkyl, provided, however, that X and Y can not both represent CH₂;

R′ represents hydrogen or alkyl;

R¹ and R², independently of each other, represent a phenyl group, which phenyl group is optionally substituted with one or more substituents independently selected from the group consisting of halo, trifluoromethyl, trifluoromethoxy, cyano and alkyl; and

R⁴, R⁵, R⁶ and R⁷ independent of each other are selected from the group consisting of hydrogen, halo, trifluoromethyl, trifluoromethoxy, cyano, alkyl, alkoxy, amino, N-alkyl-amino and N,N-dialkyl-amino.

In a preferred embodiment the 2-amino benzimidazole derivative of the invention is a compound of Formula Ia or Ib, wherein R¹ and R², independently of each other, represent a phenyl group, which phenyl group is optionally substituted with one or more substituents independently selected from the group consisting of halo, trifluoromethyl, trifluoromethoxy and cyano.

In another preferred embodiment the 2-amino benzimidazole derivative of the invention is not:
1,3-Bis-[2-(2-bromo-4-chlorophenoxy)methyl]-1,3-dihydrobenzoimidazol-2-ylideneamine;
1,3-Bis-[2-(2-bromophenoxy)methyl]-1,3-dihydrobenzoimidazol-2-ylideneamine;
(1-Benzyl-1H-benzoimidazol-2-yl)-p-tolylsulfanylmethylamine; or
[1-(4-Chlorobenzyl)-1H-benzoimidazol-2-yl]-p-tolylsulfanylmethylamine.

In a third preferred embodiment the 2-amino benzimidazole derivative of the invention is not:
1-Benzyl-3-(2-p-tolyloxyethyl)-1,3-dihydrobenzoimidazol-2-ylideneamine, 1-(2-Fluorobenzyl)-3-(2-p-tolyloxyethyl)-1,3-dihydrobenzoimidazol-2-ylideneamine;
1-Benzyl-3-[2-(4-tert-butylphenoxy)ethyl]-1,3-dihydrobenzoimidazol-2-ylideneamine;
1-(4-Bromobenzyl)-3-[2-(4-bromophenoxy)ethyl]-1,3-dihydrobenzoimidazol-2-ylideneamine;
1-(4-Bromobenzyl)-3-(2-o-tolyloxyethyl)-1,3-dihydrobenzoimidazol-2-ylideneamine;
1-(2-Fluorobenzyl)-3-(2-m-tolyloxyethyl)-1,3-dihydrobenzoimidazol-2-ylideneamine;
1-(2-Fluorobenzyl)-3-(2-o-tolyloxyethyl)-1,3-dihydrobenzoimidazol-2-ylideneamine;
1-Benzyl-3-[2-(2-chlorophenoxy)ethyl]-1,3-dihydrobenzoimidazol-2-ylideneamine;
1-(3,4-Dichlorobenzyl)-3-(2-phenoxyethyl)-1,3-dihydrobenzoimidazol-2-ylideneamine;
1-(4-Bromobenzyl)-3-(2-phenoxyethyl)-1,3-dihydrobenzoimidazol-2-ylideneamine;
1-(2-Fluorobenzyl)-3-(2-phenoxyethyl)-1,3-dihydrobenzoimidazol-2-ylideneamine;
1 (2-Chlorobenzyl)-3-(2-phenoxyethyl)-1,3-dihydrobenzoimidazol-2-ylideneamine;
1-(4-tert-Butylbenzyl)-3-(2-phenoxyethyl)-1,3-dihydrobenzoimidazol-2-ylideneamine;
1-(4-Chlorobenzyl)-3-(2-phenoxyethyl)-1,3-dihydrobenzoimidazol-2-ylideneamine;
1-(4-Methylbenzyl)-3-(2-phenoxyethyl)-1,3-dihydrobenzoimidazol-2-ylideneamine;

### 1-Benzyl-3-(2-phenoxyethyl)-1,3-dihydrobenzoimidazol-2-ylideneamine; or

1-Phenethyl-3-(2-phenoxyethyl)-1,3-dihydrobenzoimidazol-2-ylideneamine.

In a fourth preferred embodiment the 2-amino benzimidazole derivative of the invention is a compound of Formula Ia:

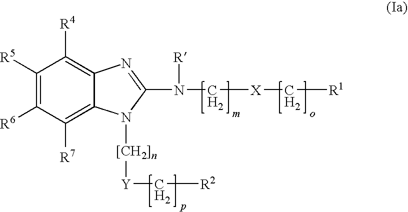

or any of its isomers or any mixture of its isomers, or a pharmaceutically acceptable salt thereof, wherein m, n, o, p, X, Y, R′, R¹, R², R⁴, R⁵, R⁶ and R⁷ are as defined above.

In a fifth preferred embodiment the 2-amino benzimidazole derivative of the invention is a compound of Formula Ib:

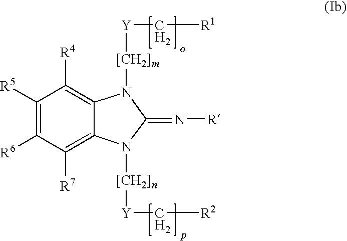

or any of its isomers or any mixture of its isomers, or a pharmaceutically acceptable salt thereof, wherein m, n, o, p, X, Y, R′, R¹, R², R⁴, R⁵, R⁶ and R⁷ are as defined above.

In a first more preferred embodiment the 2-amino benzimidazole derivative is a compound of Formula Ia or Ib, wherein m is 0, 1 or 2.

In a more preferred embodiment m is 1 or 2.

In an even more preferred embodiment m is 1.

In another more preferred embodiment m is 2.

In a sixth preferred embodiment the 2-amino benzimidazole derivative is a compound of Formula Ia or Ib, wherein n is 0, 1 or 2.

In a more preferred embodiment n is 1 or 2.

In an even more preferred embodiment n is 1.

In another more preferred embodiment n is 2.

In a seventh preferred embodiment the 2-amino benzimidazole derivative is a compound of Formula Ia or Ib, wherein o is 0, 1 or 2.

In a more preferred embodiment o is 0 or 1.

In an even more preferred embodiment o is 0.

In another more preferred embodiment o is 1.

In an eight preferred embodiment the 2-amino benzimidazole derivative is a compound of Formula Ia or Ib, wherein p is 0, 1 or 2.

In a more preferred embodiment p is 0 or 1.

In an even more preferred embodiment p is 0.

In another more preferred embodiment p is 1.

In a ninth preferred embodiment the 2-amino benzimidazole derivative is a compound of Formula Ia or Ib, wherein X and Y, independently of each other, represent CH₂, S, O or NR″; wherein R″ represents hydrogen or alkyl, provided, however, that X and Y can not both represent CH₂.

In a more preferred embodiment one of X and Y represents S or O; and the other of X and Y represents CH₂.

In an even more preferred embodiment X represents S or O; and Y represents CH₂.

In another more preferred embodiment X and Y, independently of each other, represent S, O or NR″; wherein R″ represents hydrogen or alkyl.

In a third more preferred embodiment X and Y both represent S.

In a fourth more preferred embodiment X and Y both represent 0.

In a fifth more preferred embodiment X represents S or O; and Y represents NR″; wherein R″ represents hydrogen or alkyl.

In a sixth more preferred embodiment X represents S or O; and Y represents NH.

In a seventh more preferred embodiment X represents 0; and Y represents NR″; wherein R″ represents hydrogen or alkyl.

In an eight more preferred embodiment X represents O; and Y represents NH.

In a tenth preferred embodiment the 2-amino benzimidazole derivative is a compound of Formula Ia or Ib, wherein R′ represents hydrogen or alkyl,

In a more preferred embodiment R′ represents hydrogen.

In an eleventh preferred embodiment the 2-amino benzimidazole derivative is a compound of Formula Ia or Ib, wherein R¹ and R², independently of each other, represent a phenyl group, which phenyl group is optionally substituted with one or more substituents independently selected from the group consisting of halo, in particular fluoro and chloro, trifluoromethyl, trifluoromethoxy, cyano and alkyl.

In a more preferred embodiment R¹ and R², independently of each other, represent a phenyl group, which phenyl group is optionally substituted with one or more substituents independently selected from the group consisting of halo, in particular fluoro and chloro, trifluoromethyl, trifluoromethoxy or cyano.

In an even more preferred embodiment R¹ and R², independently of each other, represent halo-substituted phenyl.

In a still more preferred embodiment R¹ and R², independently of each other, represent 4-halo-substituted phenyl.

In another more preferred embodiment R¹ and R² both represent halo-substituted phenyl.

In an even more preferred embodiment R¹ and R² both represent 4-halo-substituted phenyl.

In a yet more preferred embodiment R¹ and R² both represent a 4-fluoro or 4-chloro substituted phenyl.

In a third more preferred embodiment R¹ and R², independently of each other, represent dihalo-substituted phenyl.

In an even more preferred embodiment R¹ and R², independently of each other, represent 3,4-dihalo-substituted phenyl.

In a twelfth preferred embodiment the 2-amino benzimidazole derivative is a compound of Formula Ia or Ib, wherein R⁴, R⁵, R⁶ and R⁷ independent of each other are selected from the group consisting of hydrogen, halo, trifluoromethyl, trifluoromethoxy, cyano, alkyl, alkoxy, amino, N-alkyl-amino and N,N-dialkyl-amino. In a more preferred embodiment R⁴, R⁵, R⁶ and R⁷, independently of each other, are selected from the group consisting of hydrogen, halo, trifluoromethyl, trifluoromethoxy, cyano, alkyl and alkoxy.

In an even more preferred embodiment R⁴, R⁵, R⁶ and R⁷, independently of each other, are selected from the group consisting of hydrogen, halo, trifluoromethyl or trifluoromethoxy.

In another more preferred embodiment one of R⁴, R⁵, R⁶ and R⁷ represents halo, trifluoromethyl or trifluoromethoxy; and the others of R⁴, R⁵, R⁶ and R⁷ represent hydrogen.

In an even more preferred embodiment R⁴, R⁵, R⁶ and R⁷ all represent hydrogen.

In a thirteenth preferred embodiment the 2-amino benzimidazole derivative is a compound of Formula Ia or Ib, wherein m is 1 or 2; n is 1 or 2; o is 0 or 1; p is 0 or 1; X and Y, independently of each other, represent S or O; R′ represents hydrogen; R¹ and R² both represent halo-substituted phenyl; and R⁴, R⁵, R⁶ and R⁷ all represent hydrogen.

In a most preferred embodiment the 2-amino benzimidazole derivative is
[2-(4-Fluorophenoxy)ethyl]-{1-[2-(4-fluorophenoxy)ethyl]-1H-benzoimidazol-2-yl}amine;
1,3-Bis-[2-(4-fluorophenoxy)ethyl]-1,3-dihydrobenzoimidazol-2-ylideneamine;
1,3-Bis-(4-chlorophenylsulfanylmethyl)-1,3-dihydrobenzoimidazol-2-ylideneamine;
1,3-Bis-(4-chlorophenoxymethyl)-1,3-dihydrobenzoimidazol-2-ylideneamine; or
1,3-Bis-benzyloxymethyl-1,3-dihydrobenzoimidazol-2-ylideneamine;

or a pharmaceutically acceptable salt thereof.

Any combination of two or more of the embodiments as described above is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contains of from one to six carbon atoms (C₁₋₆-alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl is as defined above. Examples of preferred alkoxy groups of the invention include methoxy, ethoxy and isopropoxy.

In the context of this invention an N-alkyl-amino group designates a (secondary) amino group, mono-substituted with an alkyl group as defined above.

In the context of this invention an N₇N-dialkyl-amino group designates a (tertiary) amino group, di-substituted with alkyl groups as defined above.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds of the present invention may contain one or more chiral centers, and that such compounds exist in the form of isomers.

Moreover, the chemical compounds of the present invention may exist as enantiomers in (+) and (−) forms as well as in racemic forms (i). The racemates of these isomers and the individual isomers themselves are within the scope of the present invention.

The invention includes all such isomers and any mixtures thereof including racemic mixtures.

Racemic forms can be resolved into the optical antipodes by known methods and techniques. One way of separating the isomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or l- (tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo 6 receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

Compounds of the invention may be tested for their ability to modulate SK channels in vitro. Functional modulation can be determined by measuring the compound-induced change in SK current by the patch clamp technique as described in Strøbæk et al: “Inhibitory gating modulation of small conductance Ca²⁺-activated K⁺ channels by the synthetic compound (R)—N-(benzimidazol-2-yl)-1,2,3,4-tetrahydro-1-naphtylamine (NS8593) reduces afterhyperpolarizing current in hippocampal CA1 neurons”; Mol. Pharmacol. 2006 70 (5) 1771-1782. From this type of measurements the potency of a given compound can be determined as e.g. K_{d} or IC₅₀ values for blockers/inhibitors and EC₅₀ values for openers/activators. Similar data can be obtained from other patch clamp configurations and from channels expressed endogenously in various cell lines.

In one embodiment, the compounds of the invention show selectivity for SK3 over SK1 and SK2. In a further embodiment, the compounds of the invention are positive SK channel modulators, such as positive SK3 channel modulators. In a still further embodiment, the compounds of the invention are negative modulators, such as negative SK3 channel modulators. In a special embodiment, the compounds of the invention are SK channel blockers, such as SK3 channel blockers.

Based on the activity observed in the patch clamp experiments, the compound of the invention is considered useful for the treatment, prevention or alleviation of a disease or a disorder or a condition of a mammal, including a human, which disease, disorder or condition is responsive to modulation of SK channels.

In a special embodiment, the compounds of the invention are considered useful for the treatment, prevention or alleviation of absence seizures, agerelated memory loss, Alzheimer's disease, angina pectoris, arrhythmia, asthma, anxiety, ataxia, attention deficits, baldness, bipolar disorder, bladder hyperexcitability, bladder outflow obstruction, bladder spasms, brain tumors, cerebral ischaemia, chronic obstructive pulmonary disease, cancer, cardiovascular disorders, cognitive dysfunction, colitis, constipation, convulsions, coronary artery spasms, coronary hearth disease, cystic fibrosis, dementia, depression, diabetes type II, dysmenorrhoea, epilepsy, gastrointestinal dysfunction, gastroesophageal reflux disorder, gastrointestinal hypomotility disorders gastrointestinal motility insufficiency, hearing loss, hyperinsulinemia, hypertension, immune suppression, inflammatory bowel disease, inflammatory pain, intermittent claudication, irritable bowel syndrome, ischaemia, ischaemic hearth disease, learning deficiencies, male erectile dysfunction, manic depression, memory deficits, migraine, mood disorders, motor neuron diseases, myokymia, myotonic dystrophy, myotonic muscle dystrophia, narcolepsy, neuropathic pain, pain, Parkinson's disease, polycystic kidney disease, postoperative ileus, premature labour, psychosis, psychotic disorders, renal disorders, Reynaud's disease, rhinorrhoea, secretory diarrhoea, seizures, Sjorgren's syndrome, sleep apnea, spasticity, sleeping disorders, stroke, traumatic brain injury, trigeminal neuralgia, urinary incontinence, urinogenital disorders, vascular spasms, vision loss, and xerostomia.

In another preferred embodiment the compounds of the invention are considered useful for the treatment, prevention or alleviation of depression, pseudodementia, Ganser's syndrome, obsessive compulsive disorder, panic disorder, memory deficits, memory loss, attention deficit hyperactivity disorder, obesity, anxiety, eating disorder, Parkinson's disease, parkinsonism, dementia, dementia of ageing, senile dementia, acquired immunodeficiency syndrome dementia complex, memory dysfunction in ageing, social phobia, drug addiction, drug misuse, cocaine abuse, tobacco abuse, alcoholism, pain, migraine pain, bulimia, premenstrual syndrome, late luteal phase syndrome, post-traumatic syndrome, chronic fatigue syndrome, premature ejaculation, erectile difficulty, anorexia nervosa, sleep disorders, autism, mutism, trichotillomania, narcolepsy, Gilles de la Tourettes disease, inflammatory bowel disease or irritable bowel syndrome.

In yet another preferred embodiment the compounds of the invention are considered useful for the treatment, prevention or alleviation of depression, pseudodementia, Ganser's syndrome, obsessive compulsive disorders, panic disorders, memory deficits, attention deficit hyperactivity disorder, obesity, anxiety, an eating disorder or Parkinson's disease.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the submicromolar and micromolar range, i.e. of from below 1 to about 100 μM.

### Pharmaceutical Compositions

In another aspect the invention provides novel pharmaceutical compositions comprising a therapeutically effective amount of the chemical compound of the invention.

In a preferred embodiment the 2-amino benzimidazole derivative for use in a pharmaceutical composition according of the invention is not:
1,3-Bis-[2-(2-bromo-4-chlorophenoxy)methyl]-1,3-dihydrobenzoimidazol-2-ylideneamine;
1,3-Bis-[2-(2-bromophenoxy)methyl]-1,3-dihydrobenzoimidazol-2-ylideneamine;

(1-Benzyl-1H-benzoimidazol-2-yl)-p-tolylsulfanylmethylamine; or
[1-(4-Chlorobenzyl)-1H-benzoimidazol-2-yl]-p-tolylsulfanylmethylamine.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth, pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

Methods of Therapy

In another aspect the invention provides a method for the treatment, prevention or alleviation of a disease or a disorder or a condition of a living animal body, including a human, which disease, disorder or condition is responsive to modulation of SK channels, and which method comprises administering to such a living animal body, including a human, in need thereof an effective amount of a chemical compound of the invention.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

### EXAMPLES

The invention is further illustrated with reference to the following examples, which are not intended to be in any way limiting to the scope of the invention as claimed.

General: The procedures represent generic procedures used to prepare compounds of the invention. Abbreviations used are as follows:

Ac: acetyl

DMSO: dimethylsulfoxide

DMF: dimethylformamide

Et: ethyl

eq. equivalents

HR-MS: high resolution mass spectrometry

LC-MS: Liquid chromatography mass spectrometry

rt: room temperature

### Procedure A

2-Aminobenzimidazole and K₂CO₃ (4 eq) dissolved in dry acetonitrile was (under N₂) added the required aryl alkyl halide (2 eq) and stirred at 50° C. overnight. After cooling to rt, water was added and the mixture extracted with EtOAc. The combined organic phases were dried (MgSO₄), filtered and concentrated in vacuo to give the crude product which was purified by preparative LC-MS or, alternatively, by column chromatography and/or recrystallization.

An example of Procedure A, the preparation of 1,3-bis-(4-chlorophenoxymethyl)-1,3-dihydrobenzoimidazol-2-ylideneamine, is shown in Scheme 1.

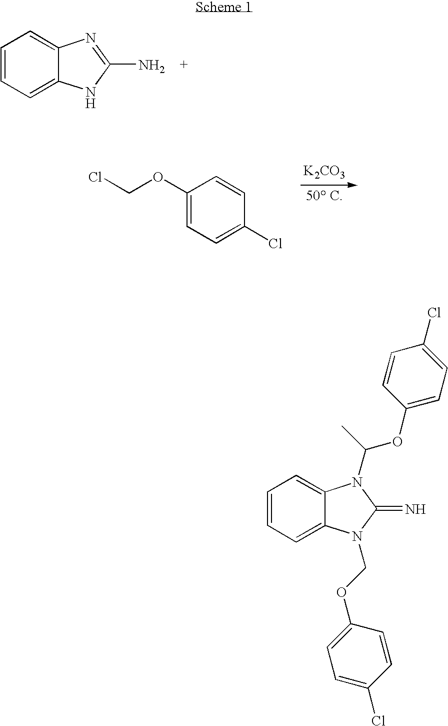

Procedure B

2-Aminobenzimidazole and K₂CO₃ (2 eq) dissolved in dry acetonitrile was (under N₂) added the required aryl alkyl halide (1 eq) and stirred at 50° C. overnight. After cooling to rt, water was added and the mixture extracted with EtOAc. The combined organic phases were dried (MgSO₄), filtered and concentrated in vacuo to give the crude N-alkylated 2-aminobenzimidazole which was purified by preparative LC-MS or, alternatively, by column chromatography.

This intermediate was subsequently, in acetonitrile or DMF and under a nitrogen atmosphere, cooled on ice, added NaH (1.2 eq) and allowed to heat to rt. To the reaction mixture was added the required substituted aryl alkyl halide and stirring was continued at room temperature over night. Water was added and the mixture extracted with EtOAc. The combined organic phases were dried (MgSO₄), filtered and concentrated in vacuo to give the crude dialkylated 2-aminobenzimidazole which was purified by preparative LC-MS or, alternatively, by column chromatography.

An example of Procedure B, the preparation of [2-(4-fluorophenoxy)ethyl]-{1-[2-(4-fluorophenoxy)ethyl]-1H-benzoimidazol-2-yl}amine, is shown in Scheme 2.

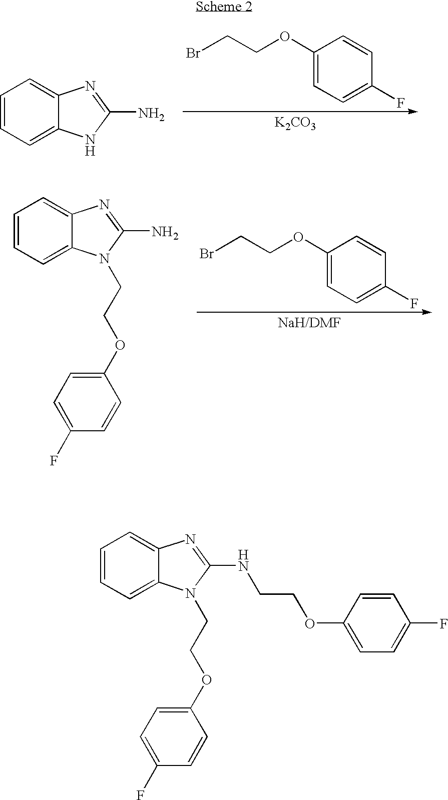

### Example 1

### [2-(4-Fluorophenoxy)ethyl]-(1-[2-(4-fluorophenoxy)ethyl]-1H-benzoimidazol-2-yl}amine

The title compound was prepared in two steps from 2-aminobenzimidazole and 1-(2-bromoethoxy)-4-fluorobenzene as described in Procedure B. The crude product was purified by preparative LC-MS to give the title compound as the free base (yellowish solid). MS (ES⁺) m/z 410 ([M+1]⁺, 100); HR-MS: 410.169900 ([M+1]⁺, C₂₃H₂₂F₂N₃O₂; calc. 410.168008).

### Example 2

### 1,3-Bis-[2-(4-fluorophenoxy)ethyl]-1,3-dihydrobenzoimidazol-2-ylideneamine

The title compound was prepared from 2-aminobenzimidazole and 1-(2-bromoethoxy)-4-fluorobenzene by Procedure A. The crude product was purified by preparative LC-MS to give the title compound as a hydrobromide salt (white solid). MS (ES⁺) m/z 410 ([M+1]⁺, 100); HR-MS: 410.170000 ([M+1]⁺, C₂₃H₂₂F₂N₃O₂; calc. 410.168008).

### Example 3

### 1,3-Bis-(4-chlorophenylsulfanylmethyl)-1,3-dihydrobenzoimidazol-2-ylideneamine

The title compound was prepared from 2-aminobenzimidazole and chloromethyl-4-chlorophenylsulfide by Procedure A. The crude product was purified by column chromatography to give the title compound as the free base (yellowish oil). ¹NMR (DMSO-d6) δ 5.48 (s, 4H), 6.86-6.89 (m, 4H), 7.34-7.36 (m, 4H), 7.45-7.59 (m, 4H); MS (ES⁺) m/z 446 (M⁺, 100).

### Example 4

### 1,3-Bis-(4-chlorophenoxymethyl)-1,3-dihydrobenzoimidazol-2-ylideneamine

The title compound was prepared from 2-aminobenzimidazole and α-4-dichloroanisole by Procedure A. The crude product was purified by preparative LC-MS to give the title compound as the free base (white solid). ¹NMR (DMSO-d6) δ 5.93 (br s, 2H), 6.01 (br s, 2H), 6.54 (s, 1H), 7.00-7.08 (m, 2H), 7.10-7.18 (m, 4H), 7.23-7.31 (m, 2H), 7.37-7.43 (m, 4H); MS (ES⁺) m/z 414 (M⁺, 100).

### Example 5

### 1,3-Bis-benzyloxymethyl-1,3-dihydrobenzoimidazol-2-ylideneamine

The title compound was prepared from 2-aminobenzimidazole and benzylchloromethylether by Procedure A. The crude product was purified by preparative LC-MS, treated with HCl in methanol and precipitated to give the title compound as a hydrochloride salt (white solid). ¹NMR (DMSO-d6) δ 4.66 (s, 4H), 5.80 (s, 4H), 7.24-7.32 (m, 10H), 7.36-7.41 (m, 2H), 7.63-7.66 (m, 2H), 9.57 (s, 2H); MS (ES⁺) m/z 374 ([M+1]⁺, 100).

### Example 6

### Biological Activity

This example demonstrates the biological activity of a compound representative of the invention. The ionic current through small-conductance Ca²⁺-activated K⁺ channels (SK channels, subtype 3) was recorded using the whole-cell configuration of the patch-clamp technique.

HEK293 tissue culture cells expressing hSK3 channels were grown in DMEM (Dulbecco's Modified Eagle Medium) supplemented with 10% FCS (foetal calf serum) at 37° C. in 5% CO₂. At 60-80% confluency, cells were harvested by trypsin treatment and seeded on cover slips.

Cells plated on coverslips were placed in a 15 μl perfusion chamber (flowrate ˜1 ml/min) mounted on an inverted microscope placed on a vibration-free table in a grounded Faraday cage. The experiments were performed at room temperature (20-22° C.). The EPC-9 patch-clamp amplifier (HEKA-electronics, Lambrect, Germany) was connected to a Macintosh computer via an ITC16 interface. Data were stored directly on the hard-disk and analysed by IGOR software (Wavemetrics, Lake Oswega, Oreg., USA).

The whole-cell configuration of the patch-clamp technique was applied. In short: The tip of a borosilicate pipette (resistance 2-4 MΩ) is gently placed on the cell membrane using remote control systems. Light suction results in the formation of a giga seal (pipette resistance increases to more than 1 GO) and the cell membrane underneath the pipette is then ruptured by more powerful suction. Cell capacitance was electronically compensated and the resistance between the pipette and the cell interior (the series resistance, Rs) was measured and compensated for. The cell capacitances ranged from 5 to 20 pF, and the series resistance was in the range 3 to 6 MΩ. Rs—as well as capacitance compensation were updated during the experiments (before each stimulus). Leak-subtractions were not performed.

The extracellular (bath) solution contained (in mM): 156 KCl, 0.1 CaCl₂, 3 MgCl₂, 10 HEPES (pH=7.4 with KOH). The test compound was dissolved in DMSO and then diluted at least 1000 times in the extracellular solution.

The intracellular (pipette) solution contained: 154 mM KCl, 10 mM HEPES, 10 mM EGTA. Concentrations of CaCl₂ and MgCl₂ needed to obtain the desired free concentrations of Ca²⁺ (0.3-0.4 μM, Mg²⁺ always 1 mM) were calculated by EqCal software (Cambridge, UK) and added.

After establishment of the whole-cell configuration, voltage-ramps (−80 to +80 mV) were applied to the cell every 5 seconds from a holding potential of 0 mV. A stable baseline current was obtained within a period of 100-500 seconds, and the compound was then added by changing to an extracellular solution containing the test compound. Activity was quantified from the change in current at −75 mV.

For inhibitors a K_{d} value, defined as the concentration required for decreasing the baseline current to 50% of the initial current, was estimated. In this assay the compounds of the invention showed K_{d} values in the sub-micromolar range (i.e. below 1 μM), which is an indication of their strong SK3 inhibiting properties.

1-15. (canceled)

16. A 2-amino benzimidazole derivative of Formula Ia or Ib: 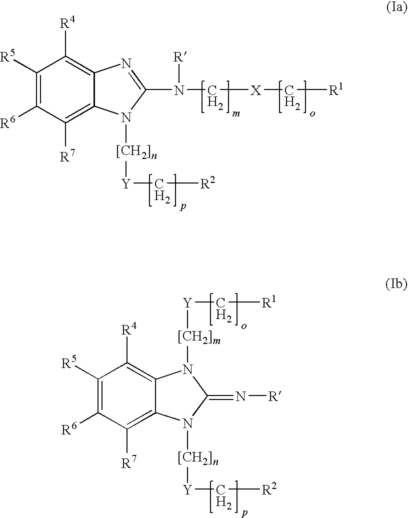
or any of its stereoisomers or any mixture of its stereoisomers, or a pharmaceutically acceptable salt thereof, wherein
m is 0, 1 or 2;
n is 0, 1 or 2;
o is 0, 1 or 2;
p is 0, 1 or 2;
X and Y, independently of each other, represent CH₂, S, O or NR″; wherein R″ represents hydrogen or alkyl provided, however, that X and Y can not both represent CH₂;
R′ represents hydrogen or alkyl;
R¹ and R², independently of each other, represent a phenyl group, which phenyl group is optionally substituted with one or more substituents independently selected from the group consisting of halo, trifluoromethyl, trifluoromethoxy, cyano and alkyl; and
R⁴, R⁵, R⁶ and R⁷ independent of each other are selected from the group consisting of hydrogen, halo, trifluoromethyl, trifluoromethoxy, cyano, alkyl, alkoxy, amino, N-alkyl-amino and N,N-dialkyl-amino.

17. The 2-amino benzimidazole derivative of claim 16, or any of its stereoisomers or any mixture of its stereoisomers, or a pharmaceutically acceptable salt thereof, wherein m is 0, 1 or 2.

18. The 2-amino benzimidazole derivative of claim 16, or any of its stereoisomers or any mixture of its stereoisomers, or a pharmaceutically acceptable salt thereof, wherein n is 0, 1 or 2.

19. The 2-amino benzimidazole derivative of claim 16, or any of its stereoisomers or any mixture of its stereoisomers, or a pharmaceutically acceptable salt thereof, wherein 0 is 0, 1 or 2.

20. The 2-amino benzimidazole derivative of claim 16, or any of its stereoisomers or any mixture of its stereoisomers, or a pharmaceutically acceptable salt thereof, wherein p is 0, 1 or 2.

21. The 2-amino benzimidazole derivative of claim 16, or any of its stereoisomers or any mixture of its stereoisomers, or a pharmaceutically acceptable salt thereof,
wherein X and Y, independently of each other, represent CH₂, S, O or NR″; wherein R″ represents hydrogen or alkyl, provided, however, that X and Y can not both represent CH₂.

22. The 2-amino benzimidazole derivative of claim 16, or any of its stereoisomers or any mixture of its stereoisomers, or a pharmaceutically acceptable salt thereof, wherein R′ represents hydrogen or alkyl.

23. The 2-amino benzimidazole derivative of claim 16, or any of its stereoisomers or any mixture of its stereoisomers, or a pharmaceutically acceptable salt thereof,
wherein R¹ and R², independently of each other, represent a phenyl group, which phenyl group is optionally substituted with one or more substituents independently selected from the group consisting of halo, trifluoromethyl, trifluoromethoxy, cyano and alkyl.

24. The 2-amino benzimidazole derivative of claim 16, or any of its stereoisomers or any mixture of its stereoisomers, or a pharmaceutically acceptable salt thereof, wherein R⁴, R⁵, R⁶ and R⁷ independent of each other are selected from the group consisting of hydrogen, halo, trifluoromethyl, trifluoromethoxy, cyano, alkyl, alkoxy, amino, N-alkyl-amino and N,N-dialkyl-amino.

25. The 2-amino benzimidazole derivative of claim 16, which is
[2-(4-Fluorophenoxy)ethyl]-{1-[2-(4-fluorophenoxy)ethyl]-1H-benzoimidazol-2-yl}amine;
1,3-Bis-[2-(4-fluorophenoxy)ethyl]-1,3-dihydrobenzoimidazol-2-ylideneamine;
1,3-Bis-(4-chlorophenylsulfanylmethyl)-1,3-dihydrobenzoimidazol-2-ylideneamine;
1,3-Bis-(4-chlorophenoxymethyl)-1,3-dihydrobenzoimidazol-2-ylideneamine; or
1,3-Bis-benzyloxymethyl-1,3-dihydrobenzoimidazol-2-ylideneamine;
or a pharmaceutically acceptable salt thereof.

26. A pharmaceutical composition, comprising a therapeutically effective amount of a 2-amino benzimidazole derivative of claim 16, or any of its stereoisomers or any mixture of its stereoisomers, or a pharmaceutically acceptable salt thereof, together with at least one pharmaceutically acceptable carrier, excipient or diluent.

27. A method for treatment, prevention or alleviation of a disease or a disorder or a condition of a living animal body, including a human, which disorder, disease or condition is responsive to modulation of SK channels, which method comprises the step of administering to such a living animal body in need thereof a therapeutically effective amount of a 2-amino benzimidazole derivative according to claim 16, or any of its stereoisomers or any mixture of its stereoisomers, or a pharmaceutically acceptable salt thereof.

28. The method according to claim 27, wherein the disease, disorder or condition responsive to modulation of SK channels is: absence seizures, agerelated memory loss, Alzheimer's disease, angina pectoris, arrhythmia, asthma, anxiety, ataxia, attention deficits, baldness, bipolar disorder, bladder hyperexcitability, bladder outflow obstruction, bladder spasms, brain tumors, cerebral ischaemia, chronic obstructive pulmonary disease, cancer, cardiovascular disorders, cognitive dysfunction, colitis, constipation, convulsions, coronary artery spasms, coronary hearth disease, cystic fibrosis, dementia, depression, diabetes type II, dysmenorrhoea, epilepsy, gastrointestinal dysfunction, gastroesophageal reflux disorder, gastrointestinal hypomotility disorders gastrointestinal motility insufficiency, hearing loss, hyperinsulinemia, hypertension, immune suppression, inflammatory bowel disease, inflammatory pain, intermittent claudication, irritable bowel syndrome, ischaemia, ischaemic hearth disease, learning deficiencies, male erectile dysfunction, manic depression, memory deficits, migraine, mood disorders, motor neuron diseases, myokymia, myotonic dystrophy, myotonic muscle dystrophia, narcolepsy, neuropathic pain, pain, Parkinson's disease, polycystic kidney disease, postoperative ileus, premature labour, psychosis, psychotic disorders, renal disorders, Reynaud's disease, rhinorrhoea, secretory diarrhoea, seizures, Sjorgren's syndrome, sleep apnea, spasticity, sleeping disorders, stroke, traumatic brain injury, trigeminal neuralgia, urinary incontinence, urinogenital disorders, vascular spasms, vision loss, or xerostomia.
